(19) United States
(12) Patent Application Publication
(10) Pub. No.: US 2012/0077977 A1
(43) Pub. Date: Mar. 29, 2012

(54) PROCESS FOR PREPARING (METH)ACRYLIC ESTERS OF N,N-SUBSTITUTED AMINO ALCOHOLS

(75) Inventors: Virginie Bette, Mannheim (DE); Hermann Bergmann, Singapore (SG); Jochen Petzoldt, Weisenheim am Berg (DE); Frank Höfer, Bad Duerkheim (DE)

(73) Assignee: BASF SE, Ludwigshafen (DE)

(21) Appl. No.: 13/236,893

(22) Filed: Sep. 20, 2011

Related U.S. Application Data
(60) Provisional application No. 61/385,572, filed on Sep. 23, 2010.

(51) Int. Cl. C07C 67/02; C07D 295/185; C07D 295/15

(52) U.S. Cl. 544/171; 560/217; 546/248

(57) ABSTRACT

The present invention relates to a process for preparing (meth)acrylic esters (F) of N,N-substituted amino alcohols, by transesterifying N,N-substituted amino alcohols (I)

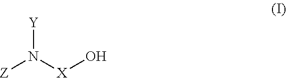

in which Y and Z are each independently C₁-C₂₀-alkyl, C₃-C₁₅-cycloalkyl, aryl, or Y and Z together with the nitrogen atom connecting them form a 5- to 9-membered saturated heterocyclic radical which optionally has oxygen, sulfur, nitrogen or C₁-C₄-alkyl-substituted nitrogen as a further heteroatom, and X is C₂-C₂₀-alkylene which may be interrupted by 1 to 10 nonadjacent oxy groups and/or unsubstituted or methoxy-substituted C₁-C₄-alkylimino groups, or a C₃-C₁₅-cycloalkylene, with at least one (meth)acrylic ester (D) in the presence of at least one heterogeneous catalyst (K), wherein the heterogeneous catalyst (K) is used without any further solvent and the content of polymerization inhibitors in the reaction mixture is <450 ppm, and to the use of the (meth)acrylic esters (F) of N,N-substituted amino alcohols.

### CROSS-REFERENCES TO RELATED APPLICATIONS

The present application is the utility application based on, and claiming benefit to, U.S. Provisional Application Ser. No. 61/385,572, filed on Sep. 23, 2010.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF THE MATERIAL ON THE COMPACT DISC

Not Applicable

### BACKGROUND OF THE INVENTION

1. Field of the Invention

The present invention relates to a process for catalytically preparing (meth)acrylic esters of N,N-substituted amino alcohols, and to the use thereof.

(Meth)acrylic acid is understood in the context of the present invention to mean acrylic acid and/or methacrylic acid, and (meth)acrylic ester to mean acrylic ester and/or methacrylic ester. (Meth)acrylic esters are also referred to hereinafter as (meth)acrylates.

2. Description of the Related Art Including Information Disclosed Under 37 CFR 1.97 and 1.98

(Meth)acrylic esters are usually prepared by acid or base-catalyzed esterification of (meth)acrylic acid or transesterification of other (meth)acrylic esters with alcohols. Frequently, acids or bases are used, and so acid or base-sensitive (meth)acrylic esters cannot be prepared in a controlled manner in this way by an esterification or transesterification.

(Meth)acrylic esters of N,N-substituted amino alcohols and the preparation thereof under different conditions are known.

For instance, EP 1 399 408 A1 discloses transesterification to prepare N,N-dialkylaminoalkyl (meth)acrylates using titanium alkoxides as catalysts. The pure product is obtained by distillation after the reaction.

EP 0 118 639 A1 describes transesterification to prepare N,N-dialkylaminoalkyl (meth)acrylates using alkoxides of titanium, aluminum, zirconium, calcium and magnesium.

JP 2001187763 describes the use of organic tin oxides as a catalyst for the transesterification reaction of alkyl(meth)acrylates with various alcohols, for example N,N-dimethylaminoethanol and N,N-diethylaminoethanol. The use of dibutyltin oxide as a catalyst for the transesterification reaction to prepare N,N-dialkylaminoalkyl (meth)acrylates is likewise known from EP 0 906 902 A1. The catalyst is finally removed from the reaction mixture by distillation.

In addition, WO 03/022796 A 1 discloses the use of zirconium acetylacetate as a catalyst for the synthesis of N,N-dialkylaminoalkyl (meth)acrylates from N,N-dialkylamino alcohols and methyl methacrylate. The removal of the zirconium catalyst is likewise effected by distillation.

Furthermore, JP 06051664 and JP 6220755 disclose the use of potassium phosphate as a catalyst for the transesterification reaction of (meth)acrylates with various N,N-dialkylamino alcohols. This involves dissolving the catalyst in the particular N,N-dialkylamino alcohol or alternatively in methanol. According to the teaching of these documents, lower yields are obtained when the catalyst is introduced into the reaction mixture as a suspension. To stabilize the reaction mixture, customary polymerization inhibitors, such as phenothiazine and hydroquinone monomethyl ether, are used in amounts of 0.05-5% by weight (500-20 000 ppm). The end product is likewise removed by distillation from the remaining catalyst.

JP 06051663 discloses a similar process, except that the use of the potassium carbonate as a catalyst is described therein. The latter is first dissolved in methanol and then added to the reaction mixture. The end product is likewise purified by distillation in order to remove the catalyst after the end of the reaction.

The processes described in the prior art have some disadvantages. Some of the catalysts disclosed therein are moisture-sensitive and relatively expensive, especially for industrial use (for example titanium and tin catalysts). In addition, all documents disclose a complex purification step, with the end product always having to be removed by distillation from the catalyst.

Japanese specifications JP 06051664, JP 6220755 and JP 06051663 additionally have the disadvantage that the potassium phosphate or potassium carbonate catalysts first have to be dissolved in a suitable solvent before they are supplied to the reaction mixture. This likewise results in a distillative purification of the end product. In addition, all three documents disclose the use of polymerization inhibitors in high amounts (>500 ppm).

### BRIEF SUMMARY OF THE INVENTION

It was therefore an object of the present invention to provide a further process with which (meth)acrylic esters of N,N-substituted amino alcohols can be prepared in high conversions and high purities from simple reactants. In addition, the above-described disadvantages of the processes described in the prior art should be overcome; more particularly, the purification of the end product should be simpler or be dispensed with entirely, and the content of polymerization inhibitors should be reduced.

### BRIEF DESCRIPTION OF THE SEVERAL VIEWS OF THE DRAWINGS

Not Applicable

### DETAILED DESCRIPTION OF THE INVENTION

The object is achieved by a process for preparing (meth)acrylic esters (F) of N,N-substituted amino alcohols, by transesterifying N,N-substituted amino alcohols (I)

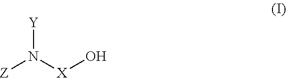

in which
Y and Z are each independently C₁-C₂₀-alkyl, C₃-C₁₅-cycloalkyl, aryl, or Y and Z together with the nitrogen atom connecting them form a 5- to 9-membered saturated heterocyclic radical which optionally has oxygen, sulfur, nitrogen or C₁-C₄-alkyl-substituted nitrogen as a further heteroatom, and
X is C₂-C₂₀-alkylene which may be interrupted by 1 to 10, preferably 1 to 5, especially 1 or 2, nonadjacent oxy groups and/or unsubstituted or methoxy-substituted C₁-C₄-alkylimino groups, or C₃-C₁₅-cycloalkylene,
   with at least one (meth)acrylic ester (D) in the presence of at least one heterogeneous catalyst (K), wherein the heterogeneous catalyst (K) is used without any further solvent and the content of polymerization inhibitors in the reaction mixture is <450 ppm, preferably <400 ppm.

With the aid of the process according to the invention, it is possible to prepare (meth)acrylic esters of N,N-substituted amino alcohols with at least one of the following advantages:
use of inexpensive reactants,
high yield,
high purity,
no complex workup (e.g. distillative removal of the end product or of solvents, removal of water).

What is particularly advantageous about the process according to the invention is the absence of suitable solvents for the catalyst. The heterogeneous catalyst (K) is suspended in the reaction mixture, such that a complex solvent removal by distillation is not required. Instead, the heterogeneous catalyst (K) can be removed from the end product by simple filtration.

In addition, it is advantageous that only a small amount of a polymerization inhibitor is used and sufficient inhibition of polymerization is nevertheless achieved.

In the specific case, the collective terms specified for the different radicals are defined as follows:

C₁-C₂₀-Alkyl: straight-chain or branched hydrocarbyl radicals having up to 20 carbon atoms, preferably C₁-C₁₀-alkyl such as methyl, ethyl, propyl, isopropyl, n-butyl, sec-butyl, tert-butyl, 1,1-dimethylethyl, pentyl, 2-methylbutyl, 1,1-dimethylpropyl, 1,2-dimethylpropyl, 2,2-dimethylpropyl, 1-ethylpropyl, hexyl, 2-methylpentyl, 3-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 2-ethylbutyl, 1,1,2-trimethylpropyl, 1,2,2-trimethylpropyl, 1-ethyl-1-methylpropyl, 1-ethyl-2-methylpropyl, heptyl, octyl, 2-ethylhexyl, 2,4,4-trimethylpentyl, 1,1,3,3-tetramethylbutyl, nonyl and decyl, and the isomers thereof.

C₃-C₁₅-Cycloalkyl: monocyclic saturated hydrocarbyl groups having 3 up to 15 carbon ring members, preferably C₃-C₈-cycloalkyl such as cyclopropyl, cyclobutyl, cyclopentyl, cyclohexyl, cycloheptyl and cyclooctyl, and a saturated or unsaturated cyclic system, for example norbornyl or norbenzyl.

Aryl: a mono- to tricyclic aromatic ring system comprising 6 to 14 carbon ring members, for example phenyl, naphthyl and anthracenyl, preferably a mono- to bicyclic, more preferably a monocyclic, aromatic ring system.

C₂-C₂₀ Alkylene which may be interrupted nonadjacent oxy groups and/or unsubstituted or methoxy-substituted C₁-C₄-alkylimino groups: ethylene, 1,2- or 1,3-propylene, 1,2-, 2,3- or 1,4-butylene, (CH₂)₂O(CH₂)₂, (CH₂)₃O(CH₂)₃, (CH₂)₂O(CH₂)₂O(CH₂)₂,

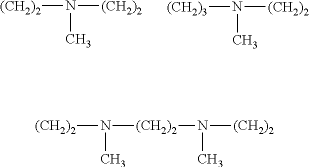

When the Y and Z radicals together with the nitrogen atom connecting them form a 5- to 9-membered, preferably 5- or 6-membered, heterocyclic radical, useful saturated heterocyclic radicals may include pyrrolidinyl, piperidinyl, morpholinyl, piperazinyl.

The substituents Y and Z may be the same or different in each case.

In a preferred embodiment, the substituents Y and Z are each independently C₁-C₁₀-alkyl, aryl, or Y and Z together with the nitrogen atom connecting them are a 5- to 9-membered heterocyclic radical which optionally has oxygen, nitrogen or C₁-C₄-alkyl-substituted nitrogen as a further heteroatom.

In a further preferred embodiment, the substituents Y and Z together with the nitrogen atom connecting them form a 5- or 6-membered heterocyclic radical which optionally has oxygen, nitrogen or C₁-C₄-alkyl-substituted nitrogen as a further heteroatom.

In a further preferred embodiment, the substituents Y and Z are each independently C₁-C₁₀-alkyl or aryl, preferably C₁-C₆-alkyl or phenyl and especially preferably C₁-C₄-alkyl or phenyl, or together form a heterocycle, preferably piperidyl or morpholyl.

In a preferred embodiment, the bridging member X is C₂-C₁₀-alkylene which may be interrupted by 1 or 5 nonadjacent oxy groups and/or unsubstituted or methoxy-substituted C₁-C₄-alkylimino groups, especially C₂-C₁₀-alkylene, preferably C₂-C₆-alkylene and especially preferably C₂-C₄-alkylene.

N,N-Substituted amino alcohols (I) suitable in accordance with the invention are, for example, N,N-dibutylaminoethanol, N,N-dimethylaminopropanol, 2-[(2-dimethylaminoethyl)methylamino]ethanol, 2-(2-dimethylaminoethoxy)ethanol, N,N-diethylaminoethanol, piperidylethanol, morpholylethanol and ethylanilineethanol.

If the N,N-substituted amino alcohols (I) are optically active, they are preferably used in racemic form or as a diastereomer mixture, but it is also possible to use them as pure enantiomers or diastereomers or as enantiomers mixtures.

General Process for Preparation of Acrylates

In the reaction step, the N,N-substituted amino alcohol (I) is transesterified with at least one, preferably exactly one, (meth)acrylic ester (D), according to the invention in the presence of at least one catalyst (K).

For the transesterification, it is possible to use (meth)acrylic esters (D) of a saturated alcohol, preferably saturated C₁-C₁₀-alkyl esters or C₃-C₁₂-cycloalkyl esters of (meth)acrylic acid, more preferably saturated C₁-C₄-alkyl esters of (meth)acrylic acid.

In the context of this document, “saturated” means compounds without C—C multiple bonds (except of course the C═C double bond in the (meth)acryloyl units).

Examples of (meth)acrylic esters (D) are methyl, ethyl, n-butyl, isobutyl, tert-butyl, n-octyl, 2-ethylhexyl and cyclohexyl (meth)acrylate, 1,2-ethylene glycol di- and mono(meth)acrylate, 1,4-butanediol di- and mono(meth)acrylate, 1,6-hexanediol di- and mono(meth)acrylate, trimethylolpropane tri(meth)acrylate and pentaerythritol tetra(meth)acrylate.

Particular preference is given to methyl, ethyl, n-butyl and 2-ethylhexyl (meth)acrylate, very particular preference to methyl, ethyl and n-butyl (meth)acrylate, particularly methyl and ethyl (meth)acrylate and especially methyl (meth)acrylate.

Catalysts (K) usable in accordance with the invention are heterogeneous catalysts which are suspended in the reaction mixture without the use of further solvents.

In this document, heterogeneous catalysts are those which have a solubility in the reaction medium at 25° C. of not more than 1 g/l, preferably of not more than 0.5 g/l and more preferably of not more than 0.25 g/l.

The catalysts (K) used are preferably inorganic salts.

Inorganic salts usable in accordance with the invention are preferably those which have a pKB of not more than 7.0, preferably not more than 4.0 and more preferably not more than 1.0. At the same time, the pKB should not be less than 1.0, preferably not less than 1.5 and more preferably not less than 1.6. Inorganic salts usable in accordance with the invention are preferably heterogeneous inorganic salts.

The inorganic salt preferably has at least one anion selected from the group consisting of carbonate (CO₃²⁻), hydrogencarbonate (HCO₃⁻), phosphate (PO₄³⁻), hydrogenphosphate (HPO₄²⁻), dihydrogenphosphate (H₂PO₄⁻), sulfate (SO₄²⁻), sulfite (SO₃²⁻) and carboxylate (R—COO⁻) in which R is C₁-C₁₈-alkyl, or C₂-C₁₈-alkyl or C₆-C₁₂-aryl optionally interrupted by one or more oxygen and/or sulfur atoms and/or one or more substituted or unsubstituted imino groups.

Preference is given to carbonate and phosphate, particular preference to phosphate.

Phosphate is also understood to mean the condensation products, for example diphosphates, triphosphates and polyphosphates.

The inorganic salt preferably has at least one, more preferably exactly one, cation selected from the group consisting of alkali metals, alkaline earth metals, ammonium, cerium, iron, manganese, chromium, molybdenum, cobalt, nickel and zinc.

Preference is given to alkali metals and particular preference to lithium, sodium or potassium.

It will be appreciated that the inorganic salts can be used in anhydrous form or in the form of their hydrates. However, they are preferably used in anhydrous form.

Particularly preferred inorganic salts are Li₃PO₄, K₃PO₄, Na₃PO₄, K₂CO₃ and Na₂CO₃ and the hydrates thereof, very particular preference being given to K₃PO₄.

According to the invention, K₃PO₄ can be used in anhydrous form, and as the tri-, hepta- or nonahydrate.

The catalyzed transesterification is effected generally at 30 to 140° C., preferably at 30 to 100° C., more preferably at 40 to 90° C. and most preferably at 50 to 80° C.

The reaction can optionally be performed under gentle vacuum of, for example, 200 hPa up to standard pressure, preferably 200 to 600 hPa and more preferably 250 to 500 hPa, if the low-boiling alcohol formed in the transesterification is to be distilled off, optionally as an azeotrope.

The molar ratio between (meth)acrylic ester (D) and N,N-substituted amino alcohol (I) is, in the transesterification catalyzed by one of the abovementioned catalyst (K), generally 1:1 to 10:1 mol/mol, preferably 1:1 to 5:1 mol/mol and more preferably 1:1 to 4:1 mol/mol.

The reaction time in the transesterification catalyzed in accordance with the invention is generally 45 min to 18 hours, preferably 2 hours to 12 hours and more preferably 3 to 10 hours.

The content of catalyst (K) in the reaction medium is generally in the range from about 0.01 to 10 mol %, preferably 0.1 to 3.0 mol % and more preferably 0.3 to 2.0 mol %, based on the sum of the N,N-substituted amino alcohols (I) used.

In the transesterification, polymerization inhibitors (as described below) are absolutely necessary.

The presence of oxygenous gases (see below) during the performance of the process according to the invention is preferred.

In the inventive transesterification, products are generally obtained with a color number below 500 APHA, preferably below 200 and more preferably below 150 (to DIN ISO 6271).

The reaction can proceed in organic solvents or mixtures thereof, or without addition of solvents. The mixtures are generally substantially anhydrous (i.e. water content below 10%, preferably below 5%, more preferably below 1% and most preferably below 0.5% by weight).

Suitable organic solvents are those known for these purposes, for example tertiary monools such as C₃-C₆-alcohols, preferably tert-butanol, tert-amyl alcohol, pyridine, poly-C₁-C₄-alkylene glycol di-C₁-C₄-alkyl ethers, preferably polyethylene glycol di-C₁-C₄-alkyl ethers, for example 1,2-dimethoxyethane, diethylene glycol dimethyl ether, polyethylene glycol dimethyl ether 500, C₁-C₄-alkylene carbonates, especially propylene carbonate, C₃-C₆-alkyl acetates, especially tert-butyl acetate, THF, toluene, 1,3-dioxolane, acetone, isobutyl methyl ketone, ethyl methyl ketone, 1,4-dioxane, tert-butyl methyl ether, cyclohexane, methylcyclohexane, toluene, hexane, dimethoxymethane, 1,1-dimethoxyethane, acetonitrile, and the mono- or polyphasic mixtures thereof.

Preference is given, however, to dispensing with the use of solvents.

In a particularly preferred embodiment of the transesterification, the reaction is performed in the (meth)acrylic ester (D) used as the reactant. Very particular preference is given to performing the reaction in such a way that the product (F), after the reaction has ended, is obtained as an about 10-80% by weight solution in the (meth)acrylic ester (D) used as the reactant, especially as a 20 to 50% by weight solution.

The reactants are present in the reaction medium in dissolved form, suspended as solids or in emulsion.

The reaction can be effected continuously, for example in a tubular reactor or in a stirred reactor cascade, or batchwise.

The reaction can be performed in all reactors suitable for such a reaction. Such reactors are known to those skilled in the art. Preference is given to effecting the reaction in a stirred tank reactor or a fixed bed reactor.

The reaction mixture can be mixed using any desired processes. Specific stirrer apparatuses are not required. The mixing can be effected, for example, by feeding in a gas, preferably an oxygenous gas (see below). The reaction medium may be mono- or polyphasic, and the reactants are dissolved, suspended or emulsified therein. The temperature is set to the desired value during the reaction and can, if desired, be increased or reduced during the course of the reaction.

When the reaction is performed in a fixed bed reactor, the fixed bed reactor is preferably equipped with immobilized catalyst (K), in which case the reaction mixture is pumped through a column filled with the catalyst (K). It is also possible to perform the reaction in a fluidized bed reactor, in which case the catalyst (K) is used immobilized on a support. The reaction mixture can be pumped continuously through the column, the flow rate being usable to control the residence time and hence the desired conversion. It is also possible to pump the reaction mixture through a column in circulation, in which case it is also possible to simultaneously distill off the alcohol released under reduced pressure.

Alcohols which are released from the (meth)acrylic esters (D) in the course of a transesterification are removed continuously or stepwise in a manner known per se, for example by means of reduced pressure, azeotropic removal, stripping, absorption, pervaporation and diffusion through membranes, or extraction.

The stripping can be effected, for example, by passing an oxygenous gas, preferably air or an air-nitrogen mixture, through the reaction mixture, optionally in addition to a distillation.

Suitable options for absorption are preferably molecular sieves or zeolites (pore size, for example, in the range of about 3-10 angstrom), removal by distillation or with the aid of suitable semipermeable membranes.

However, it is also possible to feed the removed mixture of (meth)acrylic ester (D) and the parent alcohol thereof, which frequently forms an azeotrope, directly into a plant for preparing the (meth)acrylic ester (D), in order to reutilize it therein in an esterification with (meth)acrylic acid.

After the reaction has ended, the reaction mixture obtained from the transesterification can be used without further purification, or it can be purified if required in a further step.

In general, in a purification step, only the catalyst used is removed from the reaction mixture, and the reaction product is removed from any organic solvent used.

Heterogeneous catalysts are generally removed by filtration, electrofiltration, absorption, centrifugation or decantation. The heterogeneous catalyst removed can subsequently be used for further reactions.

Preferably, in the first purification step, however, only the catalyst and any solvent used are removed.

The optionally purified reaction mixture is optionally subjected to a distillation in which the (meth)acrylic ester (F) of the N,N-substituted amino alcohols is separated by distillation from unconverted (meth)acrylic ester (D) and any by-products formed.

The distillation units are usually rectification columns of customary design with circulation evaporator and condenser. The feed is preferably into the bottom region, where the bottom temperature is, for example, 80 to 160° C., preferably 100 to 140° C., the top temperature is preferably 80 to 120° C., and the top pressure is 3 to 20 and preferably 3 to 5 mbar. It will be appreciated that the person skilled in the art can also find other temperature and pressure ranges in which the particular (meth)acrylic esters (F) of the N,N-substituted amino alcohols can be purified by distillation. What is essential is the separation of the desired product from reactants and by-products under conditions under which the desired product is subjected to a minimum level of degradation reactions.

The distillation unit has generally 5 to 50 theoretical plates.

The distillation units are of a design known per se and have the customary internals. Useful column internals in principle include all common internals, for example trays, structured packings and/or random packings. Among the trays, preference is given to bubble-cap trays, sieve trays, valve trays, Thormann trays and/or dual-flow trays; among the random packings, preference is given to those comprising rings, coils, saddles, Raschig, Intos or Pall rings, barrel or Intalox saddles, Top-Pak, etc, or braids.

The desired product is preferably distilled batchwise, in which case low boilers are first removed from the reaction mixture, and usually solvent or unconverted (meth)acrylic ester (D). After these low boilers have been removed, the distillation temperature is increased and/or the vacuum is reduced, and the desired product is distilled off.

The distillation residue remaining is usually discarded.

In addition, the end product can be purified and obtained by adsorption, for example by adsorption by means of alumina, activated carbon, silica or other adsorbents known to those skilled in the art.

The reaction conditions in the inventive transesterification are mild. Due to the low temperatures and other mild conditions, the formation of by-products in the reaction is avoided, which otherwise, for example, as a result of unwanted free-radical polymerization of the (meth)acrylic ester (D) used, which can otherwise be prevented only by addition of polymerization inhibitors.

In the inventive reaction regime, over and above the polymerization inhibitors present in the (meth)acrylic ester (D) in any case, additional polymerization inhibitors can be added to the reaction mixture, for example hydroquinone monomethyl ether, phenothiazine, phenols, for example 2-tert-butyl-4-methylphenol, 6-tert-butyl-2,4-dimethylphenol or N-oxyls such as 4-hydroxy-2,2,6,6-tetramethylpiperidine N-oxyl, 4-oxo-2,2,6,6-tetramethylpiperidine N-oxyl or Uvinul® 4040P from BASF Aktiengesellschaft, though the total amount of <450 ppm must not be exceeded in accordance with the invention. Advantageously, the transesterification is performed in the presence of an oxygenous gas, preferably air or air-nitrogen mixtures.

These polymerization inhibitors can be used individually or in any desired mixture. It is essential to the invention, however, that the content of polymerization inhibitor is not more than 450 ppm, preferably not more than 400 ppm, especially not more than 380 ppm and more preferably not more than 350 ppm. This is of significance especially for the further use of the (meth)acrylic ester (F).

The (meth)acrylic esters (F) of N,N-substituted amino alcohols (I), prepared in accordance with the invention, find use, for example, as monomers or comonomers in the production of dispersions, for example acrylic dispersions, as reactive diluents, such as in radiation-curable coating materials or in paints, and in dispersions for use in the paper sector, in the cosmetic sector, in the pharmaceutical sector, in agrochemical formulations, in the textile industry and in the oil production sector.

The examples which follow are intended to illustrate the properties of the invention, but without limiting it.

### EXAMPLES

“Parts” in this document are understood to mean “parts by weight”, unless stated otherwise.

### Example 1

The transesterification was effected in a 750 ml miniplant reactor with Oldershaw column with liquid distributor, internal thermometer, gas inlet and anchor stirrer. The return ratio was 25:1 (return stream:output stream), the stirrer speed of the anchor stirrer was 300 rpm and the air introduction rate was 1.5 l/h.

This apparatus was initially charged with 0.19 g (350 ppm) of hydroquinone monomethyl ether, 600 g (6.0 mol) of methyl methacrylate (MMA) and 165.3 g (1.0 mol) of N-ethyl-N-hydroxyethylaniline, and also 4.25 g (2.0 mol %, based on N-ethyl-N-hydroxyethylaniline) of anhydrous potassium phosphate, which were stirred. The vacuum was established (300 mbar) and the suspension was heated gradually to 70° C. During the reaction, distillate (MMA/methanol) was removed continuously and recycled according to the return ratio. The temperature in the bottom was between 70 and 75° C.; the temperature at the top of the column was between 38 and 66° C. The reaction was monitored by means of GC analysis. After 300 min, the bath temperature was reduced to 60° C. and unconverted MMA was distilled off. The reaction was ended and the vacuum was broken. The suspension was cooled and then the remaining catalyst was filtered off using a pressure suction filter.

A clear solution was obtained in a yield of 90% of ethylanilineethyl methacrylate with a purity of >98%.

### Example 2

The transesterification was effected in the apparatus described in example 1.

This apparatus was initially charged with 0.30 g (350 ppm) of hydroquinone monomethyl ether, 600 g (6.0 mol) of methyl methacrylate (MMA) and 259.9 g (1.5 mol) of N,N-dibutylaminoethanol and also 6.37 g (2.0 mol % based on N,N-dibutylaminoethanol) of anhydrous potassium phosphate, which were stirred. The vacuum was established (300 mbar) and the suspension was heated gradually to 70° C. During the reaction, distillate (MMA/methanol) was removed continuously and recycled according to the return ratio. The temperature in the bottom was between 70 and 76° C.; the temperature at the top of the column was between 38 and 66° C. The reaction was monitored by means of GC analysis. After 340 min, the bath temperature was reduced to 60° C. and unconverted MMA was distilled off. The reaction was ended and the vacuum was broken. The suspension was cooled and then the remaining catalyst was filtered off using a pressure suction filter.

A clear solution was obtained in a yield of 95% of N,N-dibutylaminoethyl methacrylate with a purity of >90%.

### Example 3

The transesterification was effected in the apparatus described in example 1.

This apparatus was initially charged with 0.25 g (350 ppm) of hydroquinone monomethyl ether, 600 g (6.0 mol) of methyl methacrylate (MMA) and 219.5 g (1.5 mol) of 2-(2-dimethylaminoethylamino)ethanol and also 6.37 g (2.0 mol % based on 2-(2-dimethylaminoethylamino)ethanol) of anhydrous potassium phosphate, which were stirred. The vacuum was established (300 mbar) and the suspension was heated gradually to 70° C. During the reaction, distillate (MMA/methanol) was removed continuously and recycled according to the return ratio. The temperature in the bottom was between 70 and 76° C.; the temperature at the top of the column was between 38 and 66° C. The reaction was monitored by means of GC analysis. After 240 min, the bath temperature was reduced to 60° C. and unconverted MMA was distilled off. The reaction was ended and the vacuum was broken. The suspension was cooled and then the remaining catalyst was filtered off using a pressure suction filter.

A clear solution was obtained in a yield of 95% of 2-(2-dimethylaminoethylamino)ethyl methacrylate with a purity of >97%.

### Example 4

The transesterification was effected in the apparatus described in example 1.

This apparatus was initially charged with 0.28 g (350 ppm) of hydroquinone monomethyl ether, 600 g (6.0 mol) of methyl methacrylate (MMA) and 193.8 g (1.5 mol) of hydroxyethylpiperidine and also 6.37 g (2.0 mol % based on hydroxyethylpiperidine) of anhydrous potassium phosphate, which were stirred. The vacuum was established (300 mbar) and the suspension was heated gradually to 70° C. During the reaction, distillate (MMA/methanol) was removed continuously and recycled according to the return ratio. The temperature in the bottom was between 70 and 76° C.; the temperature at the top of the column was between 38 and 66° C. The reaction was monitored by means of GC analysis. After 270 min, the bath temperature was reduced to 60° C. and unconverted MMA was distilled off. The reaction was ended and the vacuum was broken. The suspension was cooled and then the remaining catalyst was filtered off using a pressure suction filter.

A clear solution was obtained in a yield of >99% of piperidinylethyl methacrylate with a purity of approx. 90%. The solution still comprised 5% of the alcohol.

The remaining alcohol was removed from the product by distillation (0.1 mbar, bottom temperature 86° C.).

The end product was obtained in a yield of 86% and with a purity of >98%.

### Example 5

The transesterification was effected in the apparatus described in example 1.

This apparatus was initially charged with 0.28 g (350 ppm) of hydroquinone monomethyl ether, 600 g (6.0 mol) of methyl methacrylate (MMA) and 196.7 g (1.5 mol) of hydroxyethylmorpholine and also 6.37 g (2.0 mol % based on hydroxyethylmorpholine) of anhydrous potassium phosphate, which were stirred. The vacuum was established (300 mbar) and the suspension was heated gradually to 70° C. During the reaction, distillate (MMA/methanol) was removed continuously and recycled according to the return ratio. The temperature in the bottom was between 70 and 76° C.; the temperature at the top of the column was between 38 and 66° C. The reaction was monitored by means of GC analysis. After 360 min, the bath temperature was reduced to 60° C. and unconverted MMA was distilled off. The reaction was ended and the vacuum was broken. The suspension was cooled and then the remaining catalyst was filtered off using a pressure suction filter.

A clear solution was obtained in a yield of 86.5% of morpholinoethyl methacrylate with a purity of >92%. The solution still comprised 3.7% of the alcohol.

The remaining alcohol was removed from the product by distillation (0.1 mbar, bottom temperature 86° C.).

The end product was obtained in a yield of 86% and with a purity of >98%.

### Example 6

The transesterification was performed in a heatable 41 jacketed reactor with anchor stirrer, internal thermometer, gas inlet, column (structured Sulzer CY packing, 9 theoretical plates) and liquid divider.

A solution of 1.06 g (350 ppm) of hydroquinone monomethyl ether (MeHQ) in 2000 g (20 mol) of methyl methacrylate (MMA) was initially charged. 1032 g (10 mol) of dimethylaminopropanol and 42.45 g of anhydrous potassium phosphate were added and heated to boiling (jacket temperature (95° C.) with introduction of air (1.5 l/h) under a vacuum of 300 mbar. A reflux ratio of 10:1, then 5:1 (after 15 min), then 2:1 (after 60 min) and finally 5:1 again (after 90 min) was established. The resulting azeotrope of methanol and MMA was distilled off. The bottom temperature during the reaction was 70-90° C., the top temperature 33-63° C. After 360 min, the mixture was cooled to 64° C. and unconverted MMA was distilled off. The suspension was cooled and filtered through a pressure suction filter.

1517 g of the product were obtained in a purity of 97%.

1. A process for preparing (meth)acrylic esters of a N,N-substituted amino alcohol, the process comprising:
transesterifying the N,N-substituted amino alcohol (I) 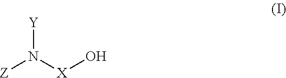
wherein
Y and Z are each independently a C₁-C₂₀-alkyl, a C₃-C₁₅-cycloalkyl, an aryl, or Y and Z together with the nitrogen atom connecting them form a 5- to 9-membered saturated heterocyclic radical, and
X is a C₂-C₂₀-alkylene,
with at least one (meth)acrylic ester (D) in the presence of at least one heterogeneous catalyst (K), wherein the heterogeneous catalyst (K) is employed without any further solvent and the content of a polymerization inhibitor in the reaction mixture is ≦450 ppm.

2. The process of claim 1, wherein the substituents Y and Z are each independently a C₁-C₁₀-alkyl, an aryl, or Y and Z together with the nitrogen atom connecting them are a 5- to 9-membered heterocyclic radical.

3. The process of claim 2, wherein the substituents Y and Z are each independently C₁-C₆-alkyl or phenyl.

4. The process of claim 1, wherein Y and Z together with the nitrogen atom connecting them are a 5- or 6-membered heterocyclic radical.

5. The process of claim 1, wherein X is a C₂-C₁₀-alkylene.

6. The process of claim 1, wherein the N,N-substituted amino alcohol (I) is selected from the group consisting of N,N-dibutylaminoethanol, N,N-dimethylaminopropanol, 2-[(2-dimethylaminoethyl)methylamino]ethanol, 2-(2-dimethylaminoethoxy)ethanol, N,N-diethylaminoethanol, piperidylethanol, morpholylethanol and ethylanilineethanol.

7. The process of claim 1, wherein the heterogeneous catalyst (K) is an inorganic salt.

8. The process of claim 7, wherein the inorganic salt has at least one anion selected from the group consisting of carbonate (CO₃²⁻), hydrogencarbonate (HCO₃⁻), phosphate (PO₄³⁻), hydrogenphosphate (HPO₄²⁻), dihydrogenphosphate (H₂PO₄⁻), sulfate (SO₄²⁻), sulfite (SO₃²⁻) and carboxylate (R—COO⁻) wherein R is a C₁-C₁₈-alkyl, or a C₂-C₁₈-alkyl or a C₆-C₁₂-aryl.

9. The process of claim 7, wherein the inorganic salt has at least one cation selected from the group consisting of an alkali metal, an alkaline earth metal, ammonium, cerium, iron, manganese, chromium, molybdenum, cobalt, nickel and zinc.

10. The process of claim 7, wherein the inorganic salt is selected from the group consisting of Li₃PO₄, K₃PO₄, Na₃PO₄ and K₂CO₃, and the hydrates thereof.

11. The process of claim 1, wherein the (meth)acrylic ester (D) is a saturated C₁₋₁₀-alkyl ester.

12. The process of claim 1, wherein the (meth)acrylic ester (D) is selected from the group consisting of methyl (meth)acrylate, ethyl (meth)acrylate, n-butyl (meth)acrylate and 2-ethylhexyl (meth)acrylate.

13. The process of claim 1, wherein the content of the polymerization inhibitor in the reaction mixture is <350 ppm.

14. The process of claim 1, wherein Y and Z together with the nitrogen atom connecting them form a 5- to 9-membered saturated heterocyclic radical comprising, as a further heteroatom, an oxygen, a sulfur, a nitrogen or a C₁-C₄-alkyl-substituted nitrogen.

15. The process of claim 1, wherein X is a C₂-C₂₀-alkylene interrupted by at least one of the following selected from the group consisting of 1 to 10 nonadjacent oxy group and an unsubstituted or methoxy-substituted C₁-C₄-alkylimino groups, or a C₃-C₁₅-cycloalkylene.

16. The process of claim 1, wherein the substituents Y and Z are each independently a C₁-C₁₀-alkyl, an aryl, or Y and Z together with the nitrogen atom connecting them are a 5- to 9-membered heterocyclic radical comprising, as a further heteroatom, an oxygen, a nitrogen or a C₁-C₄-alkyl-substituted nitrogen.

17. The process of claim 1, wherein Y and Z together with the nitrogen atom connecting them are a 5- or 6-membered heterocyclic radical comprising, as a further heteroatom, an oxygen, a nitrogen or a C₁-C₄-alkyl-substituted nitrogen.

18. The process of claim 1, wherein X is a C₂-C₁₀-alkylene interrupted by at least one of the following selected from the group consisting of 1 or 5 nonadjacent oxy groups and an unsubstituted or methoxy-substituted C₁-C₄-alkylimino group.

19. The process of claim 8, wherein the inorganic salt has at least one cation selected from the group consisting of an alkali metal, an alkaline earth metal, ammonium, cerium, iron, manganese, chromium, molybdenum, cobalt, nickel and zinc.

20. The process of claim 8, wherein the inorganic salt is selected from the group consisting of Li₃PO₄, K₃PO₄, Na₃PO₄ and K₂CO₃, and the hydrates thereof.
